(19) United States
(12) Patent Application Publication
(10) Pub. No.: US 2023/0073771 A1
(43) Pub. Date: Mar. 9, 2023

(54) SYSTEM AND METHOD FOR LABEL-FREE SINGLE MOLECULE DETECTION

(71) Applicants: Jin HE, Miami, FL (US); Popular PANDEY, Miami, FL (US)

(72) Inventors: Jin HE, Miami, FL (US); Popular PANDEY, Miami, FL (US)

(73) Assignee: The Florida International University Board of Trustees, Miami, FL (US)

(21) Appl. No.: 17/468,966

(22) Filed: Sep. 8, 2021

(51) Int. Cl. G01N 33/53; G01N 33/543; B01L 3/00; G01N 33/557; G01N 15/10

(52) U.S. Cl. CPC G01N 33/5306; G01N 33/5438; B01L 3/502715; G01N 33/557; G01N 15/1031; B01L 2300/16; G01N 2015/1006

(57) ABSTRACT

A system and method for electrical label-free detection of single protein molecules via a nanoscale electrode based on detecting the transient potential change of the floating nanoelectrode, which works for both large and small molecules. The system can also be applied to study the interactions of molecules with molecular receptors on the surface of the nanoscale electrode. The motion and dynamics of the protein near the nanoscale electrode can be detected with high precision in real time based on their intrinsic charges by the potentiometric method using a differential amplifier. The nanoelectrode can be integrated into a microfluidic device for biosensing applications.

### GOVERNMENT SUPPORT

The subject invention was made with government support under contract number 145444 awarded by the National Science Foundation. The government has certain rights in the invention.

### BACKGROUND OF THE INVENTION

Proteins are ubiquitous and play critical roles in all aspects of life. The structure, composition, and dynamics of the protein molecules determine their functions. A thorough investigation of protein molecules at the single molecule level is essential to understand their dynamic properties in an aqueous solution and provides mechanistic insight into their functions. Additionally, the single molecule study can promote the development of highly sensitive molecular diagnostic tools for biomedical applications. Due to the rapid development of precision medicine or personalized medicine, there are also high demands for revolutionary single-molecule sensor technologies in diagnostics.

Although technically challenging, several methods have been developed, such as single-entity electrochemistry methods, super-resolution optical microscopy methods, optical/magnetic tweezers, atomic force microscopy (AFM), and surface plasmon resonance microscope (SPR), to detect and characterize biomolecules and their interactions in solution or near the solution/solid interface at the single-molecule level.

However, fluorescence-based imaging and detection methods cannot detect label-free proteins. SPR can detect label-free proteins but it is still challenging to detect low molecular weight proteins. Most of the listed methods are complicated and difficult to use in portable device or clinical setting.

The electrochemical single-entity techniques have made repaid progress in recent years because they are cost-effective, relatively easy to access and use, can be conducted in portable devices, require a very small sample amount, and provide single molecule information with high throughput.

Especially, nanopore and nanoelectrode based single-entity electrochemistry methods have become increasingly popular for probing label-free biomolecules at the nanoscale. By monitoring ionic current change induced by the single-entity translocation event through the nanopore, the shape, charge, and even dynamic orientation of nanoscale entities, including proteins, can be revealed. Compared with the nanopore technique, the electrochemical nanoimpact method is another facile method to detect, characterize, and quantify nanoscale entities including biomolecules, which is based on the transduced electrochemical signal changes induced by collision events of a single-entity on the electrode surface.

The majority of the single-entity nanoimpact experiments to date are based on the amperometric method. The electrochemical current change produced during the collision events is small and often requires redox-active molecules and/or catalytically active NPs for signal amplification. As the amperometric nanoimpact experiments are often performed in the presence of foreign molecules, there is more contamination and biomolecules/nanoelectrode deactivation/passivation issues.

As an alternative method to the amperometric method in electroanalysis, the potentiometric method has been widely used in pH and ion detections by detecting the overall baseline potential change. The potentiometric method measured the potential difference between the working electrode and the reference electrode while limiting the current flow in the circuit. By using ultrasmall electrode (UME) or nanoelectrode(NE) as the working electrode, dynamic nanoimpact events by individual nanoscale entities can be sensed by the transient potential changes instead of the baseline potential change. The single-entity potentiometric sensing method is facile and versatile in that it does not require an extra signal conversion/amplification step (usually via adding redox molecules in the bath), has less contamination issues and smaller electrical noise. So far, this method has not been examined for single protein studies.

Therefore, there is a need to develop and advance the nanoimpact based potentiometric method for effective detection of individual biomolecules, such as proteins and peptides at a single molecule level, in particular, in a label-free condition.

### BRIEF SUMMARY OF THE INVENTION

The subject invention provides systems, devices and methods for single molecule detection. In one embodiment, the method for single molecule detection is label-free. The system of the subject invention provides a single-entity potentiometric method for detecting the collision events of biomolecules on a nanoelectrode. The method of the subject invention is easy to perform, highly sensitive and cost-effective.

In one embodiment, the method of the subject invention is used to probe the surface charge of individual proteins in solution by using potentiometric nanoimpact detection based on the carbon nanoelectrode (CNE) fabricated from a nanopipette. By monitoring the local potential changes during a single protein nanoimpact event near the floating CNE of a nanopipette, this method can detect various proteins, down to small proteins of about 10 kDa, and determine their charge polarity. The dynamic behavior of the protein during the approach and withdraw motions of a collision event can also be revealed based on the time-resolved potential changes and their time derivatives.

In one embodiment, the subject invention provides a method for label free detection of a biomolecule in a sample, the method comprising contacting the sample with a CNE, and measuring a signal generated from a collision event of the biomolecule on the CNE.

In one embodiment, the subject invention provides a method for a label free detection of a biomolecule in a sample, comprising:

providing a device comprising a double-barrel nanopipette sensor, the double-barrel nanopipette sensor comprising a first compartment, a second compartment, a ground electrode and a double-barrel nanopipette connecting the first and second compartment, the double-barrel nanopipette having a nanoelectrode barrel and a nanopore barrel, and the nanopore barrel being disposed with an electrode, and the first compartment being disposed with the ground electrode;

introducing the sample into the first compartment and an electrolyte solution into the second compartment that fills the nanopore barrel of the nanopipette;

optionally, applying a potential through the nanopore barrel, and

detecting the biomolecule in the sample by measuring one or more signals generated upon the interaction between the biomolecule and the nanopipette.

In one embodiment, the subject invention provides a method for detecting, quantifying and/or characterizing a biomolecule, preferably, a protein, more preferably, a charged protein, at a single-entity level in a sample solution, comprising:

providing a double-barrel nanopipette sensor comprising a double-barrel nanopipette, wherein the double-barrel nanopipette comprises a nanoelectrode barrel and a nanopore barrel, the nanopore barrel being disposed with an electrode;

contacting the double-barrel nanopipette with the sample solution;

optionally, applying a potential through the nanopore barrel, and

measuring one or more signals generated upon the interaction between the protein and the nanopipette, the one or more signals generated being a potential change (e.g., open circuit potential) of the nanoelectrode and/or an ionic current through the nanopore barrel.

In one embodiment, the subject invention provides a method of label-free detection of a protein, preferably, a charged protein, in a sample solution, comprising:

providing a double-barrel nanopipette sensor comprising a double-barrel nanopipette, wherein the double-barrel nanopipette comprises a nanoelectrode barrel and a nanopore barrel, the nanopore barrel being disposed with an electrode, and the nanoelectrode barrel being filled with a conductive material such as carbon, and gold, e.g., a carbon nanoelectrode or a gold nanoelectrode;

immersing the double-barrel nanopipette tip in the sample solution;

applying a potential through the nanopore barrel, and

detecting the protein in the sample solution at a single protein level by one or more signals generated upon the interaction between the protein and the nanopipette, the one or more signals generated being a potential change (e.g., open circuit potential) of the nanoelectrode and/or an ionic current through the nanopore barrel.

In a specific embodiment, the protein is selected from insulin, IgG, and bovine serum albumin (BSA).

In one embodiment, the method and system disclosed herein relate to rapid detection and precise quantification of molecular biomarkers in human blood, e.g., diluted human blood, and in other types of body fluids, including, serum, plasma, saliva, sweat, urine, and tear.

Also provided is the charge sensing capability of the method of the subject invention by detecting the surface charge variations of proteins at different pH. The various types of potential responses during nanoimpact events likely reflect the dynamic and inhomogeneous surface charge distribution and the flexibility of the proteins. Therefore, the potentiometric nanoimpact method is useful for the characterization of individual biomolecules and to understand their heterogeneities (such as size, charge, and shape).

The systems, devices and methods of the subject invention provide fewer contamination issues, less electrical noise and a larger detection range, and can probe various biological entities at a single-entity level. By introducing specific surface modifications to the nanoelectrode or nanopipette surface, the method can be used to probe both hit-and-run (weak nonspecific binding) and hit-and-stay (strong nonspecific binding and specific binding events) behaviors of various proteins and conduct binding kinetic analysis in complicated solution.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show the scheme of potentiometric method for a single-molecule detection using a nanopore-nanoelectrode nanopipette. (A) The schematic experimental setup of the nanopore-nanoelectrode nanopipette used to probe transient current (I) and potential (V) changes as proteins approach the nanopipette apex vicinity. The bath solution is grounded and Vpore is the bias applied to the nanopore barrel filled with 10 mM KCl solution. A high impedance differential amplifier connected to CNE is used to measure its potential change. The gradient red-colored region around the nanopipette apex represents the potential sensing zone of the nanoelectrode. The red dotted arrows denote the nanoimpact events. The black arrows on the protein molecules indicate the dipole moment direction. The top and bottom insets of (A) denote the schematic of the ionic current and potential changes due to a negatively charged protein-CNE nanoimpact event. (B) The simulated electric field (E) distribution near the nanopipette tip. The white arrows indicate the direction of the E-field, and the color bar shows the intensity of the field. The axis symmetry line is at r=0. (C) The simulated potential change (ΔV) vs. CNE-NP surface to surface distance (d) plots of uniform negatively charged (blue) and uniform positively charged (red) NP. Insets denote the simulated potential distributions around a positively and negatively charged NP (10 nm diameter; scale bar is 5 nm). (D) The fluorescence images of the nanopipette tip after immersing in phosphate buffered 10 mM KCl bath solution with 2 μM Rhodamine red labeled igG at zero Vpore and pH 4.9 (i), pH 7.2 (ii) and pH 8.5 (iii).

FIG. 2 shows the i-t and V-t time traces in control experiments without proteins in the bath solution (Vpore=0 mV). The Current (black) and potential (red) raw time traces at different bath buffer pH conditions. The baseline of V-t time traces are featureless under various buffer pH conditions.

FIGS. 3A-3E show protein-CNE nanoimpact events. (A) The electrostatic potential maps of insulin, BSA, hemoglobin and cytochrome c protein in kTe-1. The blue and red colors respectively denote the positive and negative AA residues. (B) Representative protein-CNE nanoimpact events at Vpore=0 V and pH 7.2. The potential (red) and derivative of potential (blue) time traces of insulin, BSA, hemoglobin, and cyt c. The dotted gray lines denote the potential baseline. The respective zoomed single nanoimpact event indicated by black arrows for each protein is shown in right side. (C) A typical translocation event of ferritin and cyt c. (D) Experimental result showing the percentages of the positive, negative and undecided type potential changes occurred during a single protein-CNE and nanoparticle-CNE nanoimpact events. (E) The ΔV distributions of NPs and proteins at zero Vpore. (Ferritin: N=525; −PSNP: N=195; 10 nm. GNP: N=98; 5 nm GNP: N=176; insulin: N=344; +PSNP: N=106; cyt c: N=182).

FIGS. 4A-4D show the Ferritin, BSA, cytc, Hb nanoimpact events. The Current (black) potential (red) and derivative of potential (blue) raw time traces of ferritin (a), BSA (b), Cyt c (c) and Hb (d) protein at Vpore=0 mV and pH 7.2. The nanopipettes P1, P2, P3 and P4 were used to acquire ferritin, BSA, Cyt c and HB nanoimpact events respectively.

FIGS. 5A-5C show nanoimpact events of lysozyme, HMGA2-TMR and igG-Rhodamine red. The Current (black) potential (red) and derivative of potential (blue) raw time trace of (A) lysozyme and (B) HMGA2-TMR (C) igG-Rhodamine red proteins-CNE nanoimpact events at Vpore=0 mV and in 10 mM phosphate buffered KCl with pH 7.2. Since they are positively charged at pH 7.2, dominant positive type nanoimpact events were observed. The dotted gray lines denote the potential baseline.

FIGS. 6A-6D show the nanoimpact events of polystyrene NP (PSNP) and gold NP (GNP) on the CNE surface. The potential change observed during PSNP-CNE nanoimpact events for (A) 26 nm negatively charged PSNP, (B) 50 nm positively charged PSNP (C) 10 nm negatively charged GNP and (D) 5 nm negatively charged GNP at pH 7.2. The gray, red and blue time traces respectively represent current, potential and derivative of potential (dV/dt). Dashed black lines denote the potential baseline. Over 90% of the nanoimpact events form the NP-CNE nanoimpact events are of negative type for 26 PSNP, 10 nm GNP and 5 nm GNP or positive type for 50 nm PSNP. No opposite polarity event types were observed. The potential shape and dV/dt time trace amplitudes were considered to select the nanoimpact events and the undecided nanoimpact events. The 26 nm and 50 nm PSNP respectively has ˜5% and 10% of undecided type nanoimpact events (indicated by green arrows). The respective ΔV histograms are presented in the right. The narrow ΔV histograms for NP-CNE nanoimpact events relative to proteins suggest dominant one type of nanoimpact events occur from NPs. The solid lines denote the Gaussuian fits.

FIGS. 7A-7B show the approach dV/dt histograms for ferritin and cytochrome c during translocation (blue) and collision (red) events. The dV/dt values for translocation events are always higher than that of the collision events for both proteins.

FIGS. 8A-8C show undecided nanoimpact events. The potential change observed during protein-CNE anoimpact events for (A) ferritin, (B) Hemoglobin and (C) cytochrome c at Vpore=0 mV and pH 7.2. The red and blue time traces represent potential and derivative of potential (dV/dt). The nanoimpact events denoted by blue arrows are counted while the nanoimpact events denoted as red arrows were assigned as undecided. The potential shape and dV/dt time trace amplitudes were considered to select the nanoimpact events and the undecided nanoimpact events. Typically for positive and negative nanoimpact dV/dt features a small but measurable spikes. In contrast, the undecided nanoimpact events are usually slow and do not have features in the dV/dt.

FIGS. 9A-9D show single protein charge sensing by tuning pH of the bath electrolyte solution. (A) Theoretical estimation of the net charge of Hb and cyt c at pH 7.2. (B) Plot of fraction of positive events appeared in each of the pH conditions for Hb and cyt c. N=3. The dotted lines in a, b, and d serve as eye guide. Normalized bar chart of ΔV for (C) cyt c and (D) Hb at three different pH conditions. The solid lines in the histograms are the Gaussian fits. The percentage values denote the fraction of positive type nanoimpact events observed for particular pH conditions.

FIGS. 10A-10D shows the comparison of potential signals generated by collision events of 40 nm gold nanoparticles on biphenyl-4-thiol (BPT) molecule modified GNE, CNE and bare gold nanoelectrode (GNE). (A) Molecular structure of BPT and a SEM image of a GNE. (B) The potential signals generated by collision events of 40 nm gold nanoparticles on (BPT) modified GNE (blue), CNE (green) and bare GNE (pink). The individual collision events are shown in zoomed image below. ΔV and ΔT denote the potential dip amplitude and an approach time duration respectively. (C) Time derivative of potential (dV/dt) signals in (B). (D) The histograms of ΔV and ΔT distributions of GNP collision events at the BPT modified GNE, bare CNE and bare GNE. The solid lines denote the Gaussian fits.

FIG. 11 shows the scheme of a nanoelectrode sensor in a microfluidic device for biosensing applications.

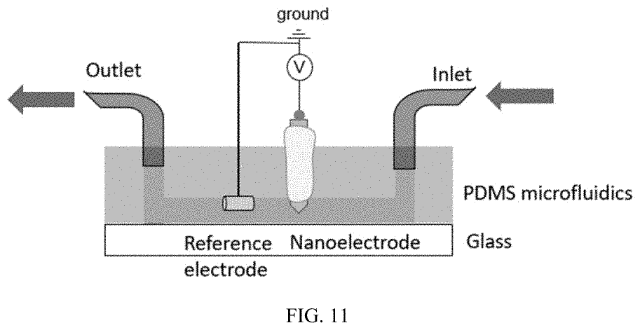

### DETAILED DESCRIPTION OF THE INVENTION

The subject invention provides systems, devices, and methods for label-free detections and analyses of biomolecules including large and small molecules, e.g., proteins and peptides, preferably in their natural form, and their dynamic interactions with substrates, e.g., conductive materials, at a single molecule level. The system can be utilized as a portable and point-of-care device. The single-molecule sensor technology can be used in the fields of, for example, the rapid developed precision medicine or personalized medicine.

In one embodiment, the systems, devices and methods relate to the field of detection of biomolecules within a sample, such as testing the presence and concentration of one or more proteins within a sample solution for the purposes of screening for diseases and conditions.

In one embodiment, the system comprises a double-barrel nanopipette sensor comprising a double-barrel nanopipette connecting two compartments or reservoirs: first and second compartments or reservoirs, each with its own electrode, a first electrode and a second electrode. In one embodiment, the double-barrel nanopipette sensor comprises two nanodetectors at the nanopipette apex.

In a preferred embodiment, the first and second compartments are liquid compartments. The first electrode and the second electrode are preferably Ag/AgCl electrodes. The disclosed nanopipette biosensor allows for the quick, low-cost quantification of biomolecules within a given sample using the principle of emerging electrochemistry single-entity techniques.

In one embodiment, the nanopipette has a tapered tip. One important advantage of the nanopipette is that it can be made cheaply and reproducibly, with a few tens of nanometer resolution, from glass or quartz capillary tubes. It is highly versatile in application and fabrication. For example, the nanopipette can be used as a nanopore sensor for chemical and biological sensing and electrophysiological applications. Owing to its tip geometry, the nanopipette can also be developed as a scanning probe for scanning ion conductance microscopy (SICM) and scanning electrochemical microscopy (SECM).

The term “quartz” means a nanopipette media is a fused silica or amorphous quartz, or crystalline quartz. Ceramics and glass ceramics and borosilicate glasses may also be utilized. The term “quartz” is intended and defined to encompass that special material as well as applicable ceramics, glass ceramics or borosilicate glasses. Various types of glass or quartz may be used in the present nanopipette fabrication. A primary consideration is the ability of the material to be drawn to a narrow diameter opening. The preferred nanopipette material consists essentially of silicon dioxide, as included in the form of various types of glass and quartz.

In a specific embodiment, the double-barrel nanopipette is fabricated from theta micropipettes. One barrel is converted to a nanoelectrode by filling the tip of the barrel with conductive materials, such as pyrolytic carbon, copper, graphite, titanium, brass, palladium, silver, platinum, gold. The other barrel remains open at the tip and can be used as a nanopore. The nanoelectrode can be made by different methods. The open barrel is suitable for holding inside of it a fluid that can be passed through the nanopore at the tip opening. The two barrels are separated by a thin wall, e.g., a quartz wall.

In one embodiment, the first compartment or reservoir is a sample compartment or reservoir receiving a sample solution containing the biomolecules for detection. The second compartment or reservoir receives a solution that fills the open barrel of the nanopipette. The first and second compartments or reservoirs are in fluid communication via the nanopore at the tip of the nanopipette. Preferably, the solution that fills the open barrel of the nanopipette is an electrolyte.

In one embodiment, the double-barrel nanopipette comprises a nanopore and a nanoelectrode at the tip. The nanopore and the nanoelectrode are very close to each other. In one embodiment, the nanoelectrode is a stable nanoscale conductive electrode, such as carbon, gold, platinum or palladium nanoelectrode. In a preferred embodiment, the nanoelectrode is a CNE made by deposition of pyrolyzed carbon in quartz nanopipette. The final CNE geometry can be controlled by the flow speed/pressure of butane (carbon source) and argon (protective gas) during carbon deposition.

In one embodiment, the first electrode in the first compartment is a reference electrode to the second electrode and the nanoelectrode. When an electric potential is applied to the sensor via the first and second electrodes in the first and second compartments, an ionic current flowing between the two compartments can be measured by a current detector. When a biomolecule moves by or collides with the nanoelectrode, a transient potential change between the nanoelectrode and the first electrode can be measured by a potential detector.

In one embodiment, the potential detector is a voltage meter. In a specific embodiment, a voltage meter with a high-input impedance is connected to the nanoelectrode for potential measurement. In a specific embodiment, the current detector is a low-noise current amplifier connected to the nanopore. In certain embodiments, the current detector is sensitive for detecting changes in current on the order of 1-100, 10-100, 20-100, 20-50, 1-20 or 1-10 picoamperes. In certain embodiments, the current detector can detect changes, in current, of at least 1, 10, 20, 30, 40, 50, 60, 70, 80, 90, or 100 picoamperes. It should have an input in a circuit where a known voltage can be supplied. Such detectors include, for example, voltage clamp amplifiers and transimpedance amplifiers.

In one embodiment, an external electric force can be applied via nanopore bias (Vpore) through the nanopore barrel, which can modulate the motion of biomolecules in the sample solution near the nanopipette tip. In some embodiments, Vpore can be, for example, between ±1V, ±900 mV, ±800 mV, 700 mV, ±600 mV, t 500 mV, ±400 mV, ±300 mV, 200 mV, 100 mV, ±50 mV, 20 mV, or ±10 mV.

In one embodiment, the first and second electrodes are not chemically modified. In one embodiment, the nanoelectrode surface is modified by recognition molecules, for example, antibodies, DNA oligomers, peptide, synthetic polymers, and small molecules with various functional groups, e.g., amine, amide, and carboxylic acid. In one embodiment, the surface of the nanopore, including outer and inner surface of the nanopore at the tip of the nanopipette, may be modified by silane molecules with various functional groups, e.g., 3-cyanopropyldimethlychlorosilane. In one embodiment, the surface modification on the nanopore, nanoelectrode, and/or nanopipette may or may not immobilize or bind to the biomolecules in the sample solution.

In one embodiment, the nanoelectrode is surface-modified with chemicals to improve protein detection specificity and sensitivity, and avoid fouling.

In one embodiment, the nanopore has a conical shape. In one embodiment, the diameter of the nanopore is from about 5 nm to about 200 nm, from about 5 nm to about 150 nm, from about 5 nm to about 125 nm, from about 5 nm to about 100 nm, from about 10 nm to about 200 nm, from about 10 nm to about 150 nm, from about 10 nm to about 100 nm, from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, from about 10 nm to about 20 nm, from about 20 nm to about 100 nm, from about 20 nm to about 90 nm, from about 20 nm to about 80 nm, from about 20 nm to about 70 nm, from about 20 nm to about 60 nm, from about 20 nm to about 50 nm, from about 20 nm to about 40 nm, or from about 20 nm to about 30 nm.

In one embodiment, the nanopore has a resistance ranging from about 0.1 to about 12 GΩ, from about 0.2 to about 11 GΩ, from about 0.5 to about 10 GΩ, from about 1 to about 10 GΩ, from about 1.5 to about 9.5 GΩ, from about 1.5 to about 9 GΩ, from about 2 to about 8.5 GΩ, from about 2 to about 8 GΩ, from about 2 to about 7.5 GΩ, from about 2 to about 7 GΩ, from about 2 to about 6.5 GΩ, from about 2 to about 6 GΩ, from about 2 to about 5.5 GΩ, from about 2 to about 5 GΩ, from about 2 to about 4.5 GΩ, from about 2 to about 4 GΩ, or from about 2 to about 3.5 GΩ.

In one embodiment, the nanoelectrode extends out of the tip slightly having an effective area from about 0.01 to about 5 μm², from about 0.02 to about 4.5 μm², from about 0.05 to about 4 μm², from about 0.1 to about 3.5 μm², from about 0.1 to about 3 μm², from about 0.1 to about 2.5 μm², from about 0.1 to about 2 μm², from about 0.1 to about 1.5 μm², from about 0.1 to about 1 μm², from about 0.1 to about 0.5 μm², from about 0.01 to about 1 μm², from about 0.01 to about 0.5 μm², from about 0.01 to about 0.2 μm², from about 0.02 to about 1 μm², from about 0.02 to about 0.5 μm², from about 0.02 to about 0.4 μm², from about 0.02 to about 0.3 μm², or from about 0.02 to about 0.2 μm².

In one embodiment, the effective diameter of the nanoelectrode ranges from about 5 nm to about 400 nm, from about 5 nm to about 300 nm, from about 5 nm to about 250 nm, from about 5 nm to about 200 nm, from about 10 nm to about 200 nm, from about 10 nm to about 150 nm, from about 10 nm to about 100 nm, from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, from about 10 nm to about 20 nm, from about 20 nm to about 100 nm, from about 20 nm to about 90 nm, from about 20 nm to about 80 nm, from about 20 nm to about 70 nm, from about 20 nm to about 60 nm, or from about 20 nm to about 50 nm.

In one embodiment, the surface of the nanopipette is negatively charged.

In one embodiment, the system is applied to study the interactions of molecules with molecular receptors on the surface of the nanoscale electrode. The motion and dynamics of the protein near the nanoscale electrode can be detected with high precision in real time based on their intrinsic charges by using a differential amplifier.

In one embodiment, the nanopipette sensor can be integrated into a microfluidic device for biosensing applications. For example, the first and second compartments may be part of, or in fluid communication with, microchannels of the device. The nanoelectrode in a microfluidic channel can be used to achieve flow sensing with controlled flow speed (e.g., via a syringe pump), which can enable higher throughput and sensitivity.

In one embodiment, the subject invention provides a microfluidic device comprising a microfluidic channel and a nanoelectrode sensor disposed in the microfluidic channel, wherein the microfluidic channel has an outlet and an inlet such that the microfluidic channel is in fluid communication with a sample. In certain embodiments, the microfluidic device is made of polydimethylsiloxane (PDMS), glass or a combination thereof.

In specific embodiments, the microfluidic device comprises a microfluidic channel in a substrate on a solid support, e.g., glass, wherein the substrate can be PDMS and materials alike, including, but are not limited to, poly(methyl methacrylate) (PMMA), polycarbonate, polystyrene, poly(ethylene glycol) diacrylate (PEGDA), cyclic olefin copolymer, and cyclic olefin polymer (COP).

In one embodiment, the subject invention provides a microfluidic device comprising a microfluidic channel and a nanoelectrode disposed in the microfluidic channel, wherein the microfluidic channel has an outlet and an inlet such that the microfluidic channel is in fluid communication with a sample.

In one embodiment, the subject invention provides a device for detecting, quantifying or characterizing a biomolecule, wherein the device comprises

a first compartment and a second compartment;

a double-barrel nanopipette, wherein a first barrel is filled with a conductive material, and a second barrel is disposed with an electrode, and wherein the double-barrel nanopipette connects the first compartment and the second compartment; and

a reference electrode disposed in the first compartment.

The microfluidic channel can have any dimension suitable for a sample solution to pass through. In some embodiments, the channel or microfluidic channel may have a height in the range of, for example, 1-10000 μm, 1-1000 μm, 1-900 μm, 1-800 μm, 1-700 μm, 1-600 μm, 1-500 μm, 1-400 μm, 1-300 μm, 1-200 μm, 1-100 μm, 5-100 μm, 10-100 μm, 10-50 μm, 10-20 μm, 1-90 μm, 1-80 μm, 1-70 μm, 1-60 μm, 1-50 μm, 1-40 μm, 1-30 μm, 1-20 μm, or 1-10 μm. In some embodiments, the channel or microfluidic channel may have a width in the range of, for example, 1-10000 μm, 1-1000 μm, 1-900 μm, 1-800 μm, 1-700 μm, 1-600 μm, 1-500 μm, 1-400 μm, 1-300 μm, 1-200 μm, 1-100 μm, 1-90 μm, 1-80 μm, 1-70 μm, 1-60 μm, 1-50 μm, 1-40 μm, 1-30 μm, 1-20 μm, or 1-10 μm. In certain embodiments, the channel or microfluidic channel length to channel or microfluidic channel depth ration may be, for example 1:1 to 150:1.

In specific embodiments, the microfluidic channel near the position of the nanoelectrode can be, for example, 1-100, 1-90, 1-80, 1-70, 1-60, 1-50, 1-40, 1-30, 1-20, or 1-20 microns in width and 1-50, 1-40, 1-30, 1-20, 5-20, or 10-20 microns in height.

In one embodiment, the device further comprises a potential detector and/or a current detector.

In one embodiment, the system and device of the subject invention can be used to measure or detect simultaneously electrochemical reactions and biomolecule-nanopipette interactions occurring at the tip of the nanopipette. The electrochemical reactions and biomolecule-nanopipette interactions include i) nanoimpact events at the nanoelectrode vicinity and ii) biomolecule translocation events through the nanopore.

In one embodiment, the nanoelectrode vicinity refers to a region or area surrounding the extended tip of the nanoelectrode, and having a distance, from about 0 to about 20 nm, from the extended tip of the nanoelectrode. Preferably, the nanoelectrode vicinity serves as a sensing zone of any nanoimpact. The sensing zone has a radius from about 0 to about 100 nm, from about 0 to about 90 nm, from about 0 to about 80 nm, from about 0 to about 70 nm, from about 0 to about 60 nm, from about 0 to about 50 nm, from about 0 to about 40 nm, from about 0 to about 30 nm, from about 0 to about 25 nm, from about 0 to about 20 nm, from about 0 to about 15 nm, from about 0 to about 12 nm, from about 0 to about 10 nm, from about 1 to about 50 nm, from about 1 to about 40 nm, from about 1 to about 30 nm, from about 1 to about 20 nm, from about 1 to about 15 nm, or from about 1 to about 10 nm.

In one embodiment, the translocation events of biomolecules through the nanopore are very sensitive to the size ratio between biomolecule and the nanopore. For example, the ratio of biomolecule to nanopore, e.g., in size, or dimension, may be 1:1.1, 1:1.2, 1:1.3, 1:1.4. 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, or any ratio therebetween.

In certain embodiments, the subject invention provides methods of detecting, diagnosing, monitoring and/or managing conditions in a subject. Detection of a biomarker/biomolecule in a sample can be indicative, or confirmatory, of a diagnosis of a condition. Monitoring can include detection or repeated detection of a biomarker/biomolecule from a sample or samples of a subject in which the biomarker/biomolecule has already been detected. Managing can include therapeutic intervention based upon the presence or absence of a biomarker/biomolecule in a subject. On the basis of the detection of the presence, absence or quantity of a biomarker/biomolecule in a subject, a treatment can be selected, administered, monitored, and/or modified. Detection of the presence, absence or quantity of a biomarker/biomolecule in a subject can comprise detection of a biomarker/biomolecule in a sample from the subject.

The detection methods described herein can be performed on a subject or on a sample from a subject. A sample can contain, or be suspected of containing, a biomarker/biomolecule. A sample can be a biological sample from a subject. The subject can be a subject having, diagnosed with, suspected of having, at risk for developing a condition, or even a subject without any indicia of the condition. In one embodiment, the sample can be a blood sample, a serum sample, a plasma sample, a cerebrospinal fluid sample, or a solid tissue sample. A sample solution is formed with a sample from a subject. In one embodiment, the sample may be cell culture medium, cell extract or cell lysate.

The subject can be an animal subject, preferably a mammal and most preferably a human. The subject may be, but are not limited to, non-human primates, rodents (e.g., rats, mice), dogs, cats, horses, cattle, pigs, sheep, goats, chickens, guinea pigs, hamsters and the like.

The method of the subject invention utilizes the principle of emerging electrochemistry single-entity techniques and a dual-channel nanopipette with both nanopore and nanoelectrode at the tip. When a charged biomolecule, such as protein, moves near the vicinity of the nanoelectrode of the nanopipette, distinct local electrostatic potential changes induced by the transient collision event of the biomolecule (e.g., protein), also called “nanoimpact” events, can be captured by the floating nanoelectrode. The variation in potential changes can be attributed to the intrinsic protein dynamics and surface charge heterogeneity based on the finite element method and molecular dynamic simulations.

Advantageously, the potentiometric method is highly sensitive for charged biomolecules, e.g., charged proteins. In specific embodiments, low molecular weight proteins less than 10 kDa can be detected using the provided system and method.

When a biomolecule, e.g., a protein molecule, in solution approaches the nanoelectrode surface and inside the double layer of the nanoelectrode, the charge of the biomolecule can disturb the potential of the floating nanoelectrode, which can be picked up by the potential detector connected to the nanoelectrode. The potential change is highly sensitive to the distance between the protein and the nanoelectrode surface. Because of the small size of the nanoelectrode, only one or a few biomolecules are very close to the carbon nanoelectrode at one time point, thus transient potential changes induced by individual biomolecule can be observed in the time-resolved potential traces.

In one embodiment, the potential detector is a high-input impedance differential amplifier (based on an instrumentation amplifier). The potential measurement is conducted at a low gain setting, 1× or 10×. Compared with the traditional low noise current amplifier, high gain setting (such as 1×10⁹) is needed to detect pA-nA current changes induced by nanoscale objects, which limiting the time resolution.

In the low gain setting, the potential amplifier can measure about 10 μV changes with a high bandwidth (at least 50 kHz, 0.02 ms). The potential sensitivity and time resolution are enough to detect the motion of individual biomolecule during their interactions with the nanoelectrode surface. For example, with 10 mM salt concentration, small proteins such as insulin (5.8 kDa) can induce about 0.1 mV potential change on a carbon nanoelectrode.

The motion of the biomolecule in solution can be driven by thermal fluctuations or external electric field. Because of the high mobility of small biomolecules, no external bias (Vpore) is normally needed to achieve high throughput detection.

In one embodiment, the subject invention provides a method for detecting a biomolecule in a sample by using the system and device of the subject invention. The method of using the system and device comprises introducing a sample solution containing the biomolecule into the first compartment or reservoir, and introducing an electrolyte solution into the second compartment or reservoir to fill the open barrel of the nanopipette, wherein the nanopore at the tip of the nanopipette connects the first compartment or reservoir to the second compartment or reservoir, enabling the biomolecules in the first compartment or reservoir to flow through the nanopore, and to move in the vicinity of the nanoelectrode. As the biomolecules flow through the nanopore and/or move in the vicinity of the nanoelectrode, ionic current and potential changes can be recorded and analyzed to provide various measured parameters of the biomolecules, including size, shape, charge, concentration and so on.

In one embodiment, the method for a label free detection, quantification and/or characterization of a biomolecule in a sample, comprises contacting the sample with a system comprising a nanoelectrode, and measuring a signal, e.g., a transient electrical potential change, induced by a collision event of the biomolecule on the nanoelectrode.

In specific embodiments, the nanoelectrode is a CNE made from a single-barrel nanopipette or double-barrel nanopipette, or electrochemically etched tip from metal wires or other nanofabrication methods known in the art. In a specific embodiment, the metal wire is, for example, copper, titanium, palladium, silver, platinum, or gold.

In one embodiment, the method for detecting, quantifying and/or characterizing a biomolecule, preferably, at a single molecule level, comprises:

providing a sample containing a biomolecule or suspected to contain the biomolecule;

contacting the sample with a nanoelectrode for measuring a potential difference or change in response to a collision event from the biomolecule at the nanoelectrode; and

measuring and/or monitoring the potential difference or change for detecting, quantifying or characterizing the biomolecule.

In one embodiment, the method for detecting, quantifying and/or characterizing a biomolecule, preferably, at a single-entity level, comprises:

providing a sample containing a biomolecule or suspected to contain the biomolecule;

adding the sample to the first compartment of the device or system of the subject invention;

adding an electrolyte to the second compartment of the device or system of the subject invention; and

detecting, quantifying or characterizing the biomolecule by measuring and/or monitoring the current and/or potential change in response to biomolecule-nanopipette interactions at the tip of the nanopipette.

In one embodiment, the method for detecting, quantifying and/or characterizing a biomolecule in a solution or a sample, preferably, at a single-entity level, comprises:

providing a double-barrel nanopipette sensor comprising a first compartment, a second compartment, and a double-barrel nanopipette that connects the first and second compartments, wherein the double-barrel nanopipette comprises a nanoelectrode and a nanopore at the tip;

introducing the solution or sample into the first compartment and an electrolyte solution into the second compartment, wherein each of the first and second compartments contains an electrode disposed to measure voltage difference, current flow or resistance between the two compartments or to apply a potential bias or an external electrical field; and

detecting, quantifying or characterizing the biomolecule by measuring and/or monitoring the current and/or potential change in response to biomolecule-nanopipette interactions at the tip of the nanopipette.

In one embodiment, the electrode disposed in the first compartment is a ground or reference electrode.

In one embodiment, the step of detecting, quantifying or characterizing the biomolecule comprises:

measuring a current flow between the two compartments over time as individual biomolecule passes through the nanopore,

measuring a potential change of the nanoelectrode over time as individual biomolecule approaching the vicinity of the nanoelectrode and bouncing back, and/or

measuring a resistance between the two compartments.

In one embodiment, the step of detecting, quantifying or characterizing the biomolecule may further comprise determining the translocation events of the biomolecule in the sample or solution, and/or through the nanopore from potential and/or ionic current measurements.

In one embodiment, the method of the subject invention may further comprise a step of applying a potential via nanopore bias through the nanopore barrel prior to any measurements.

In one embodiment, the method of the subject invention further comprises analyzing the measured current, potential and/or resistance to obtain the various parameters of the biomolecule, including size, shape, and charge.

In one embodiment, the step of detecting, quantifying and/or characterizing the biomolecule may further comprise comparing the obtained parameters to a standard, e.g., known parameters and signatures of the biomolecule.

The biomolecules can be, for example, proteins, peptides, enzymes, antibodies, DNAs, and RNAs. In some embodiments, the biomolecules are proteins having a molecular weight of at least 500 Da, at least 750 Da, at least 1 kDa, at least 5 kDa, at least 10 kDa, at least 15 kDa, at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, at least 100 kDa, at least 125 kDa, at least 150 kDa, at least 200 kDa, at least 250 kDa, at least 300 kDa, at least 350 kDa, at least 400 kDa, at least 450 kDa, at least 500 kDa or any molecular weight in between.

In specific embodiments, the proteins that can be detected include, but are not limited to, cytochrome-c (12 kDa), insulin (5.8 kDa), HMGA2, hemoglobin, IgG, bovine serum albumin (BSA), ferritin and lysozyme.

In one embodiment, the subject invention also provides a method for detecting, quantifying and/or characterizing a biomolecule in a sample solution, preferably, at a single-entity level, the method comprising:

providing a double-barrel nanopipette sensor comprising a double-barrel nanopipette, and a ground electrode, wherein the double-barrel nanopipette comprises a first barrel filled with a conductive material, i.e., nanoelectrode, and a second barrel, i.e., nanopore barrel, disposed with an electrode and back-filled with an electrolyte;

immersing the double-barrel nanopipette in the sample solution, and placing the ground electrode in the sample solution at some distance away,

optionally, applying a potential through the second barrel, and

detecting, quantifying and/or characterizing the biomolecule in the sample solution by measuring and/or monitoring the current flow through the nanopore barrel, and/or potential change (e.g., open circuit potential) in response to biomolecule-nanopipette interactions at the tip of the nanopipette.

A variety of electrolyte solutions may be used in the nanopipette. The electrolyte solutions contain dissolved electrolytes, i.e., free ions. Typical ions include sodium, potassium, calcium, magnesium, chloride, phosphate and bicarbonate. Other ionic species may be used. The electrolyte should carry an ionic current. The electrolyte solution may comprise from about 5 to about 160 mM, from about 10 to about 150 mM, from about 10 to about 140 mM, from about 10 to about 130 mM, from about 10 to about 120 mM, from about 10 to about 110 mM, from about 10 to about 100 mM, from about 10 to about 90 mM, from about 10 to about 80 mM, from about 10 to about 70 mM, from about 10 to about 60 mM, from about 10 to about 50 mM, from about 10 to about 40 mM, from about 10 to about 30 mM, or from about 10 to about 20 mM, of positive and negative ionic species.

A variety of salts may be used in the electrolyte solution. They are composed of cations (positively charged ions) and anions (negative ions) so that the product is electrically neutral (without a net charge). These component ions can be inorganic such as chloride (Cl—), as well as organic such as acetate (CH₃COO⁻) and monatomic ions such as fluoride (F—), as well as polyatomic ions such as sulfate (SO₄²⁻). There are several varieties of salts. Salts that hydrolyze to produce hydroxide ions when dissolved in water are basic salts and salts that hydrolyze to produce hydronium ions in water are acid salts. Neutral salts are those that are neither acid nor basic salts. Molten salts and solutions containing dissolved salts (e.g. sodium chloride in water) are called electrolytes, as they are able to conduct electricity.

In one embodiment, the same or different electrolyte solution may be used in the nanopipette and the sample solution. In one embodiment, the sample may be processed to obtain a sample solution comprising the same or different electrolyte as in the nanopipette. In one embodiment, the electrolyte solution is saline, such as PBS. In one embodiment, the electrolyte solution has a physiological solution comprising a salt or salts that are essentially isotonic with body fluid.

In one embodiment, the sample solution may have a pH value from 3 to 11, 3 to 10, 4 to 10, 5 to 9, or 6 to 8 or other range between 3 and 11. In one embodiment, the sample solution has a physiological pH.

In one embodiment, the method and system of the subject invention can be used to determine the charge and motion of the biomolecule, e.g., protein, from non-specific molecule-substrate surface interaction events. Such determination comprises analyzing the shape and magnitude of the recorded time-resolved potential change and its time derivative. In some embodiments, the charge variation of the biomolecule, e.g., protein, at different pH can also be determined.

In one embodiment, the subject invention provides nanoimpact based potentiometric methods effective in the detection of individual label-free proteins, including cytochrome-c (12.4 kDa), based on their native charge in aqueous solution. The proteins in solution frequently interact with the CNE of the nanopore-nanoelectrode nanopipette. The individual Hit-and-Run type of weak interaction events with the CNE surface induces small but distinct electrostatic potential changes of the floating CNE. The recorded potential changes can differentiate the polarity of charge carried by the protein and reveal the behavior of the proteins during the nanoimpact events.

In one embodiment, the method of the subject invention can be used to detect the net charge variation of a single biomolecule, e.g., protein induced by a pH change. Combined with molecular dynamic (MD) and finite element method (FEM) simulations, this variation likely originates from the intrinsic flexibility and nonuniformly distributed surface charge of the protein.

In one embodiment, the nanoelectrode can be coupled with a nanopore detector, another single-molecule sensor, to enhance the detection capability of the nanopore.

In one embodiment, the method can be used for protein binding and binding dynamic analysis, such as a binding event of the biomarker molecule to an antibody. Similar techniques for ensemble measurements are ELISA (enzyme-linked immunosorbent assay) and SPR (surface plasmon resonance).

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### EXAMPLES

### Methods

### Electrochemical Single-Entity Measurements

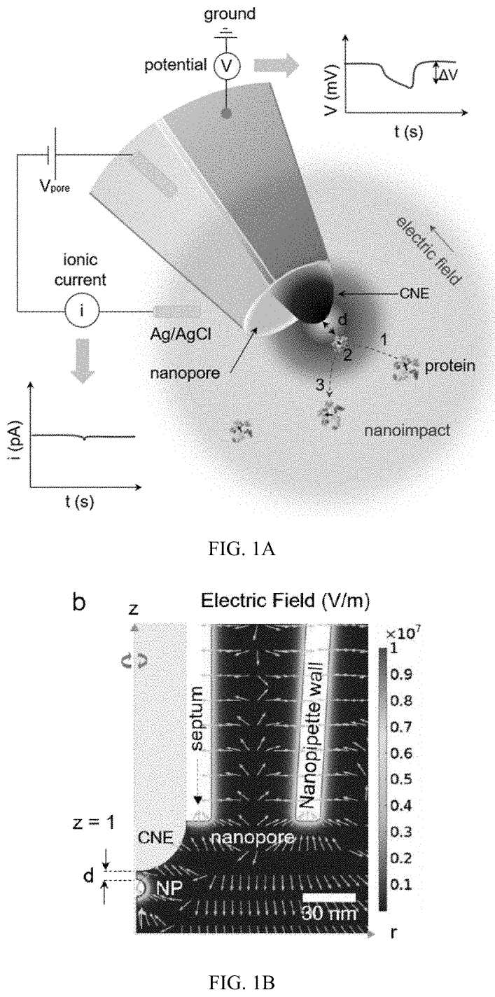

The i-t and V-t traces are recorded using the experimental setup illustrated in FIG. 1(a). The Axopatch 200B amplifier (Molecular Devices Inc., CA) is used in voltage-clamp mode to measure the ionic current. A home-built high input impedance differential amplifier is used to measure the open-circuit potential V of the nanoelectrode. An oscilloscope (Yokogawa DL850) is used to record the data with a sampling rate of 50 kHz. All the measurements are performed at room temperature. The bulk concentrations of proteins, PS NPs and GNPs in 10 mM phosphate-buffered KCl bath are typically 100-500 pM, if not mentioned otherwise.

### FEM Simulation

FEM simulation was used to solve coupled Poisson-Nernst-Planck (PNP) partial differential equations at the steady-state. Two COMSOL Multiphysics (v 5.2) modules, namely, AC/DC and chemical reaction engineering modules were used for the FEM simulation.

### Example 1—Potentiometric Nanoimpact Experimental Setup for Probing Individual Proteins

The experimental setup is illustrated in FIG. 1A. The nanopore-CNE nanopipettes used in the study (>50) have a long-taper geometry. To match the size variations of the measured nanoscale entities from small proteins to large nanoparticles, the pore diameter ranges from 10 to 50 nm and the effective surface area of CNE ranges from 0.02 to 0.56 μm². The detailed information of the nanopipettes with data presented in the study is summarized below (named P1-P15, see Table 1). Details of the fabrication and characterization of nanopore-CNE nanopipettes can be found in Pandey et al. 2019.

TABLE 1
The nanopore diameter and the effective CNE
surface area of 15 nanopipettes.

| | CNE Area | Nanopore | |
|---|---|---|---|
| Nanopipette | (μm²) ^{a} | diameter (nm)^{b} | Measurements |
| P1 | 0.02 ± 0.01 | 19 ± 2 | Insulin |
| P2 | 0.05 ± 0.01 | 28 ± 2 | BSA |
| P3 | 0.03 ± 0.01 | 18 ± 2 | ferritin |
| P4 | 0.02 ± 0.01 | 21 ± 3 | cytc |
| P5 | 0.12 ± 0.02 | 14 ± 2 | Hb |
| P6 | 0.20 ± 0.01 | 15 ± 2 | cytc |
| P7 | 0.03 ± 0.01 | 12 ± 1 | Hb |
| P8 | 0.09 ± 0.01 | 31 ± 4 | Hb |
| P9 | 0.09 ± 0.01 | 90 ± 8 | 50 nm PS NP |
| P10 | 0.56 ± 0.04 | 45 ± 4 | 26 nm PS NP |
| P11 | 0.15 ± 0.02 | 22 ± 3 | 10 nm GNP |
| P12 | 0.17 ± 0.02 | 17 ± 2 | 5 nm GNP |
| P13 | 0.11 ± 0.01 | 16 ± 2 | Lysozyme |
| P14 | 0.21 ± 0.03 | 34 ± 4 | HMGA2-TMR |
| P15 | 0.18 ± 0.02 | 29 ± 3 | IgG-Rh Red |

^{a} The error in the CNE effective area is mainly due to the uncertainly of geometry.
^{b}The error in the nanopore diameter is calculated based on the uncertainty of the nanopipette geometry (half cone angle)

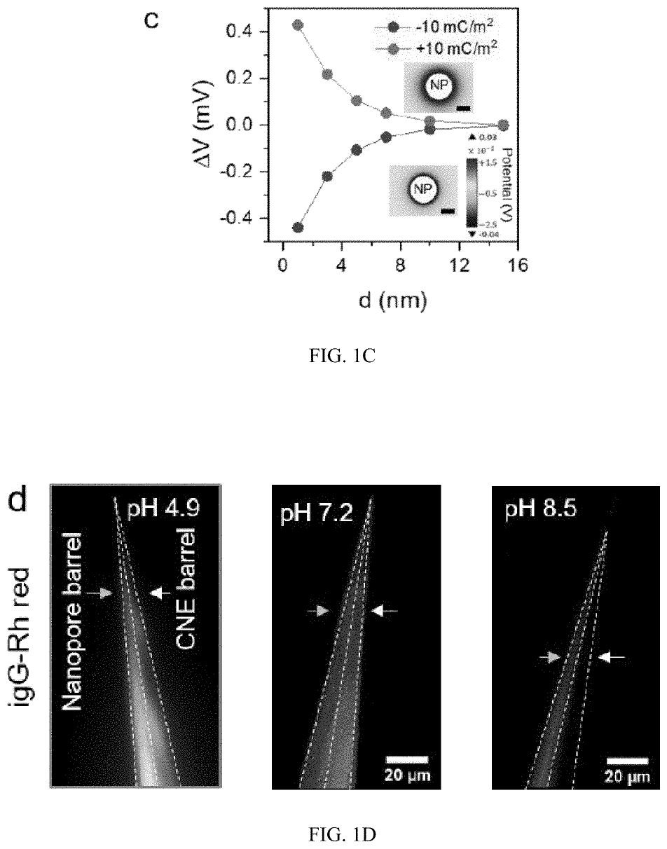

An external electric force can be applied via nanopore bias (Vpore) through the nanopore barrel to modulate the motion of proteins. Without protein molecules, in the phosphate-buffered 10 mM KCl bath solution, both the time traces of ionic current (i) and open-circuit potential (V) acquired respectively through the nanopore and CNE were stable and featureless (FIG. 2). After adding proteins for ˜2-5 min, small i and V changes appeared frequently in the time traces. These transient changes are induced by the interactions between proteins and the two electrochemical nanodetectors at the nanopipette apex and mostly are at a single-molecule level.

As shown in FIG. 1A, two types of transient protein-nanopipette interaction events may be observed: (i) nanoimpact at the CNE vicinity and (ii) translocation through the nanopore. Because of the small size of protein, the type (ii) translocation events are very sensitive to the size ratio between protein and the nanopore. In general, when the nanopore size is more than 3 times bigger than the protein, the translocation events are difficult to be detected by the ionic current. In contrast, for negatively charged proteins with bigger size, they are difficult to enter the pore due to large repelling electrostatic force. Though type (ii) events through the nanopore (10 nm<d<20 nm) can be occasionally observed, signals induced by type (i) events are dominant. In addition, different from the current signal, the potential signal of type (i) event is less sensitive to the size of CNE. The Vpore is also not effective for nanopimpact at the CNE, especially for smaller proteins with higher mobility. Zero Vpore is, thus, often used when detecting the nanoimpact events of proteins on the CNE.

To better understand how the protein surface charge affects the potential change of CNE, numerical simulations by FEM were performed implementing a 2D axial symmetry model as shown in FIG. 1B. In 10 mM KCl, a 10 nm dielectric NP with a uniform surface charge density (±10 mC/m²) was placed near a floating NE to mimic a nanoimpact event. FIG. 1B shows the distribution and direction of the electric field (E) near the NE when the distance (d) is 5 nm between the NE surface and the front surface of a negatively charged NP. The simulation results revealed that nanoimpact events change the local distributions of ion, E, and the potential V of the floating NE. When the NP is away from the NE, the floating NE is slightly negative due to the nearby negatively charged nanopipette surface. The approaching of the negatively charged NP make the NE more negative and the approaching of the positively charged NP makes the NE less negative. FIG. 1C shows the potential change (ΔV) vs. d plots for the negatively charged (blue) and positively charged (red) NPs. The ΔV magnitude is always bigger as d is decreased from 15 nm to 1 nm for both NPs with opposite charge polarities.

To shed light on the motion of the charged proteins and their interactions with the nanopipette tip, fluorescently labeled proteins were tested. In one experiment, IgG antibody (˜14 nm, 144 kDa, pKa=7.0) with rhodamine (Rh) red fluorescence tag was added to the bath solution with different pH. With pKa near 7, the IgG is positive at pH 4.9 and becomes negative at pH 8.5. Meanwhile, the quartz surface is more negative at pH 8.5 because of the silanol groups. FIG. 1D shows the fluorescence images of the nanopipette tip after immersing the tip in the bath solution for ˜30 min at zero Vpore. Generally, a weaker fluorescence signal is observed at the CNE barrel side, which is originated solely from the adsorbed proteins on the quartz surface. In contrast, the higher fluorescence at the nanopore barrel side is from both the adsorbed proteins on the quartz surface and the translocated proteins inside the barrel solution. The fluorescence intensity of the tip is reduced with the increase of pH from 4.9 to 8.5. At pH 8.5, the fluorescence intensity is obviously reduced at both barrels and can hardly be detected at the CNE barrel side. Therefore, the motion of proteins and their interactions with the CNE near the nanopipette tip is obviously influenced by the electrostatic interactions between charged protein and the negatively charged nanopipette tip. The positive protein normally has stronger adsorption to the quartz surface and is easier to translocate through the pore.

### Example 2—Potentiometric Recording of Single Protein-CNE Nanoimpact Events at pH 7.2

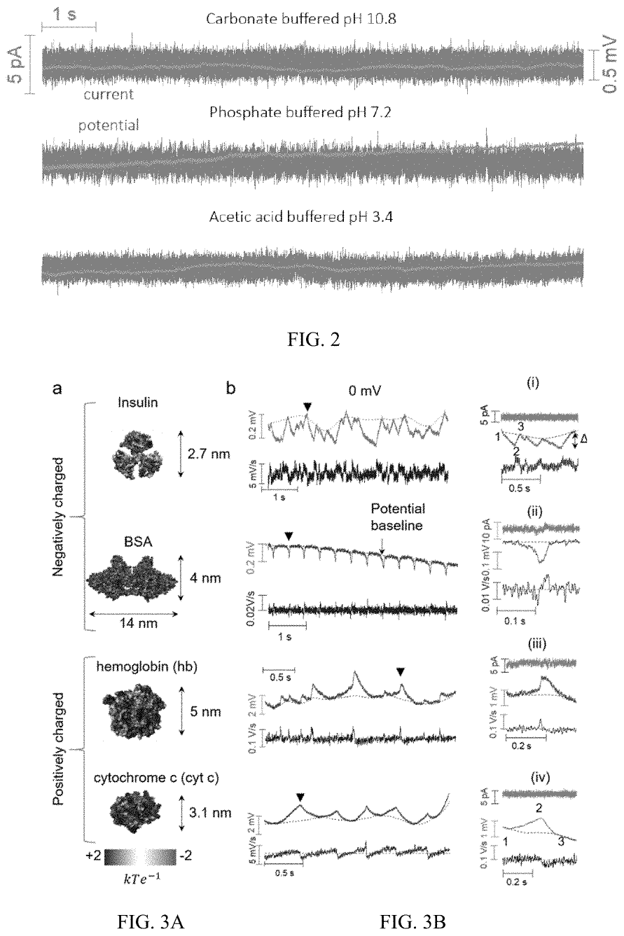

To validate the nanoimpact based potentiometric detection of single protein molecules using a nanopore-CNE nanopipette, a series of proteins were measured. FIG. 3A shows the electrostatic potential maps and size of four globular proteins, insulin, BSA (133.4 kDa), hemoglobin (Hb, 64.5 kDa), and cyt c at pH 7.2. The corresponding net surface charges of insulin, Hb and cyt c were estimated using protein calculator (v3.4) as −6.3e, −28.0e, +4.9e, and +9.2e, respectively. Other physicochemical properties of the proteins used in the study can be found in Table 2.

TABLE 2
Characteristics of the proteins used in the study

| Protein | Mol Wt. | Diameter | | Net charge @ | D |
|---|---|---|---|---|---|
| (PDB ID) | (kDa) | (~nm) | pI | pH 7.2 (e) | (D) |
| Ferritin (1IER)¹ | 440.0 | 11 | 5.4 | −72.0 | 447* |
| Hemoglobin | 64.5 | 5 | 8.7 | +3.5 | 191 |
| (2QSP)² | | | | | |
| Cytochrome c | 12.4 | 3 | 10.2 | +9.2 | 267 |
| (1HRC)³ | | | | | |
| BSA (3V03)⁴ | 133.4 | a = 14, | 5.8 | −28.0 | 852 |
| | | b = 4* | | | |
| Lysozyme | 14.4 | 2 | 9.0 | +7.6 | 272 |
| (1DPX)⁵ | | | | | |
| HMGA2⁶ | 11.8 | 3 | 11 | +13 | — |
| IgG | 144 | 14.5 | 7.0 | +2.7 | 2492 |
| Insulin (2ZP6) | 5.8 | 2 | 5.3 | −6.3 | 206 |

*Dipole moment of ferritin monomer. Dipole moment in Debye (D) of all proteins in their native state was estimated from the protein dipole moment server.⁷
BSA has an oblate structure with major axis length a = 14 nm and minor axis length b = 4 nm.⁸

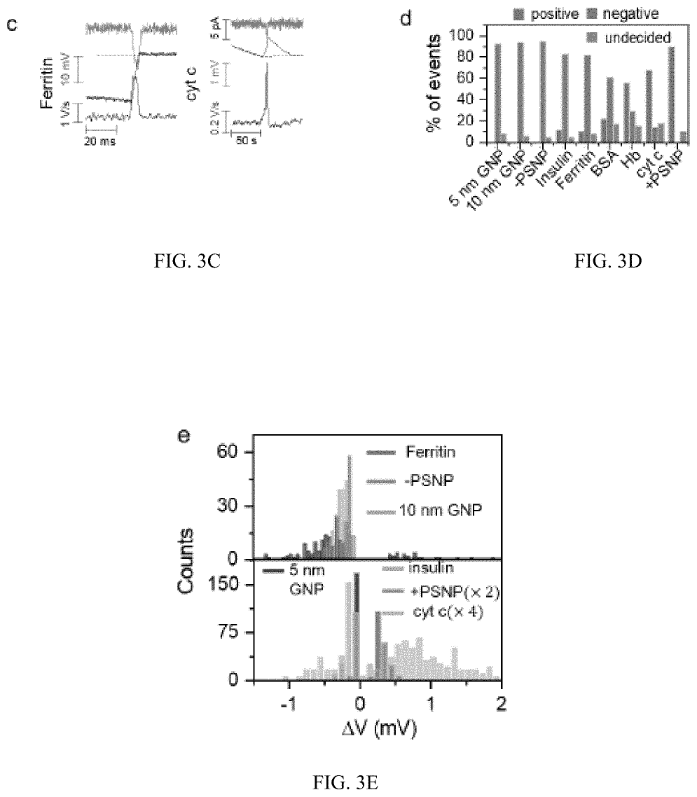

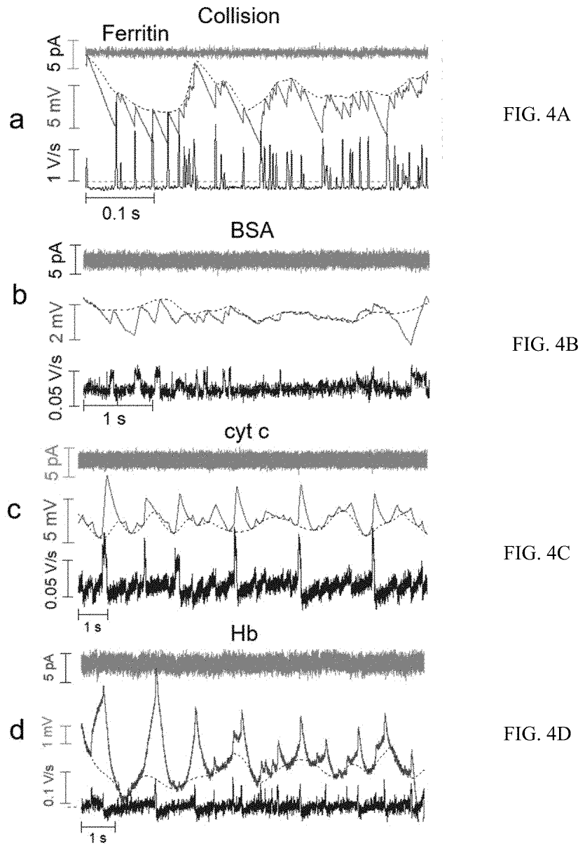

Four nanopipettes P1-P4 were used to probe insulin, BSA, Hb, and cyt c proteins, respectively, in a 10 mM phosphate-buffered KCl bath (pH=7.2) at Vpore=0 mV. FIG. 3B also shows the representative V-t time traces (red) with continuous small potential changes but negligible current changes, which are induced by nanoimpact events of negatively charged insulin (i) and BSA (ii) and positively charged cyt c (iii) and hemoglobin (iv) proteins. Additional data can be seen in FIGS. 4A-4D. Similar results are also observed for lysozyme, HMGA2 and IgG (FIGS. 5A-5C).

The respective zoomed time traces for the nanoimpact events of each protein are presented at the right column of FIG. 38. The shape reveals the overall impact motion of protein. The ΔV reflects the net charge carried by the protein while the slope (dV/dt) provides information on the speed of the protein near the CNE vicinity. For negatively charged entities, the potential signal has been well understood. As the insulin approaches the CNE (position 1→2), reaches the closest distance (position 2) and rebound (position 2-6) from the CNE, the potential change features a gradual decrease (more negative from gray potential baseline) followed by a sharp increase in potential, as shown in FIG. 3B (i). The dV/dt value appears negative (˜−0.15 V/s) during the approach and then reaches positive maxima (˜1.5 V/s) as the insulin rebounds from the CNE surface. Similar shapes of potential and dV/dt signals were observed for BSA as shown in FIG. 3B (ii). The slower approaching and faster rebounding motions of the insulin and BSA protein are likely due to the combined effect of the higher local concentration of protein near the CNE and the stronger electrostatic repulsion between the nanopipette and proteins. The potential change and the dV/dt peak of ferritin (FIGS. 4A-4D) are generally larger than that of insulin and BSA (FIG. 3B), which can be attributed to the much bigger net charge of the ferritin. Here the baseline is an eye guide, which can be determined when there are fewer events.

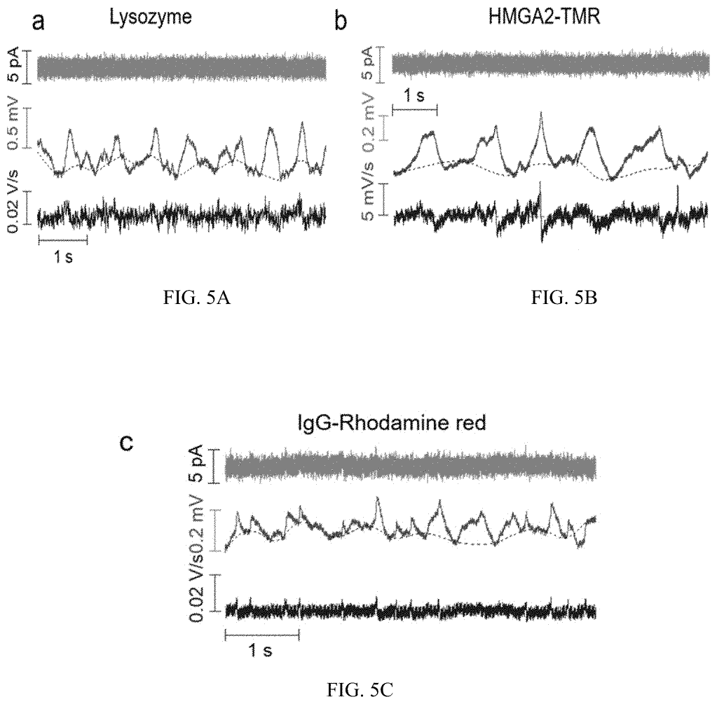

The results of positive proteins are generally noisier with bigger fluctuations than that of negatively charged proteins. About 20% of the potential changes show bigger ΔV (>5 mV) for both positively charged cyt c and Hb but less than 5% of the events show ΔV>5 mV for both negatively charged ferritin and BSA. We attribute the bigger potential changes to clustered proteins and adsorption events and thus not considered in the statistical analysis. The representative transient potential signals with small ΔV amplitude for a positively charged cyt c nanoimpact event are presented in FIG. 3B (iv). As the cyt c protein approaches (position 1 and rebounds (position 2-3) from the CNE, the potential change features a gradual increase followed by a gradual decrease in potential. The dV/dt features a small positive value (˜+5 mV/s) during the approach and a larger negative value (˜10 mV/s) during the rebound. A zoomed Hb nanoimpact event is shown in FIG. 3B (iii). The potential and dV/dt shapes are generally like that of the cyt c. The upward potential change is opposite to that of the negatively charged proteins. Similar transient potential changes were also observed for 50 nm size positively charged PSNP (FIG. 6B). The generally slower approach and rebound motions for positive proteins can be attributed to the adsorbed proteins at the nanopipette surface which repels the incoming proteins.

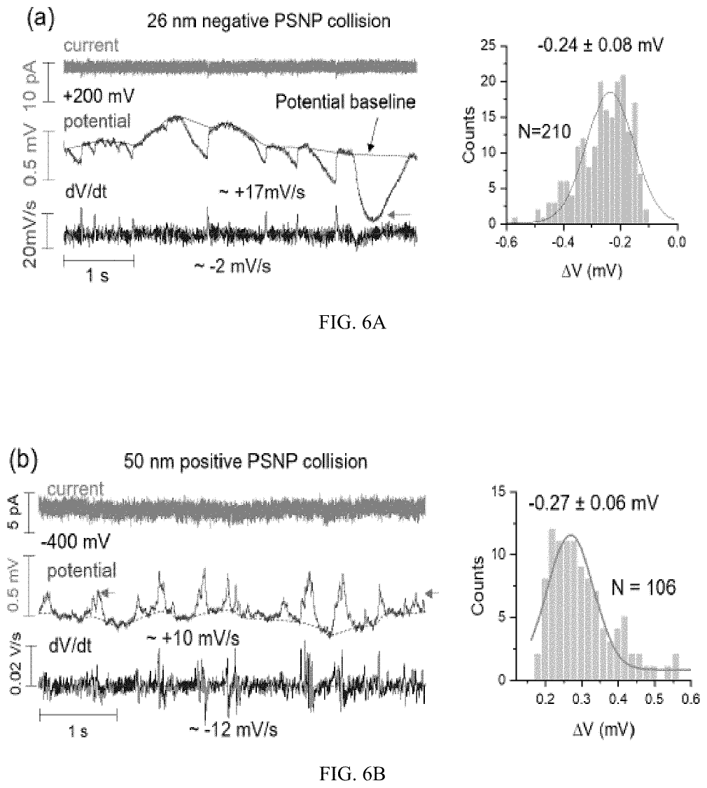

For comparison, FIG. 3C also denotes the typical ferritin and Cyt c nanopore translocation events from nanopores of P1 and P3. The detectable translocation events can be differentiated from nanoimpact events based on the two criteria: 1) Both potential and current changes can be observed simultaneously; 2) translocation events have at least 1.5 times larger positive dV/dt (i.e. translocation speed) values than the nanoimpact events (FIGS. 7A-7B). For example, the ferritin nanoimpact and the translocation events (FIG. 3C) have similar dV/dt values (˜2 V/s) and can be separated based on current change. For the smaller size cyt c, nanoimpact events do not feature big current changes. However, the dV/dt is more than 5 times smaller than the corresponding translocation event (FIG. 3C).

The shape of the potential and dV/dt time traces allows the differentiation of the positive and negative type nanoimpact events. If the dV/dt has a negative/positive approach value just before rebound back, the nanoimpact events are from the negatively/positively charged proteins as shown in zoomed nanoimpact events in FIG. 3B. Through a careful analysis of the potential and dV/dt shape, the net charge polarity of the impacting biomolecules can be revealed.

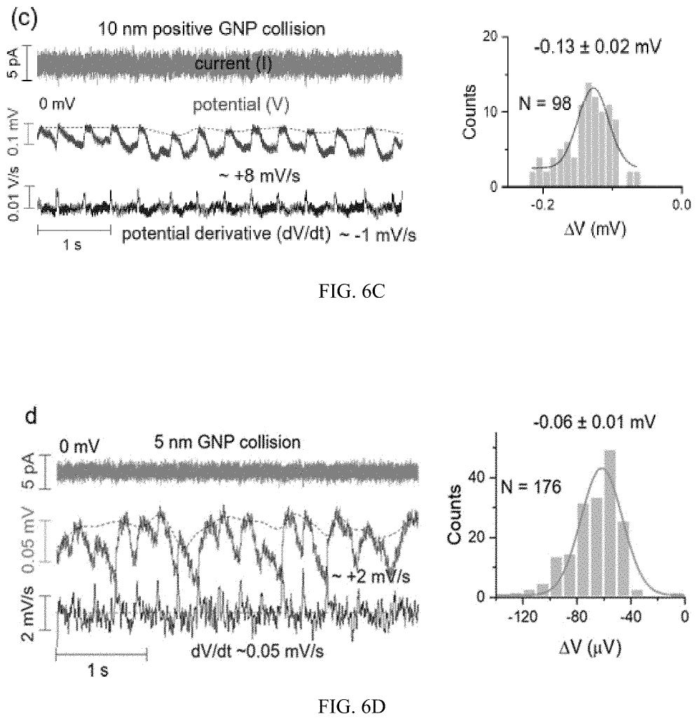

Interestingly, two populations of nanoimpact events were observed at the same pH condition: negative and positive type nanoimpact events. FIG. 3D shows the percentages of positive, negative and undecided nanoimpact events of proteins at pH 7.2. The nature of the difficult to categorize events is shown in FIGS. 8A-8C. In the experiment, the potential baseline noise is 0.05-0.1 mV range. A ΔV greater than 2× standard deviation (a) of the potential baseline noise is considered as nanoimpact events. Proteins with net negative charge always have higher percentages (insulin ˜83%, ferritin ˜82% and BSA ˜61%) of the negative type nanoimpact events. In contrast, proteins with net positive charge always have higher percentages (Hb ˜53% and Cytc ˜65%) of the positive type nanoimpact events. A small percentage of ambiguous events were also observed, which were kept as undecided (orange color). This could be due to the dynamic and inhomogeneous surface charge distribution of the protein, which will be further discussed later. For comparison, rigid and uniformly charged polystyrene nanoparticles (PSNPs), 26 nm size negatively charged and 50 nm positively charged, were also measured (FIGS. 6A-6D). No ambiguous and opposite polarity events were observed for these PSNPs.

The statistical analysis of ΔV for proteins and nanoparticles at pH 7.2 and Vpore=0 mV are presented in FIG. 3E. The ΔV is always negative for negatively charged 26 nm PSNP and 10 and 5 nm GNPs, and positive for positively charged 50 nm PSNP. Meanwhile, the ΔV of majority signals is negative for negative proteins ferritin, BSA, and insulin, and positive for positive proteins cyt c and Hb. These results support that the CNE is sensitive to changes in the overall surface charge of proteins.

Intriguingly, the ΔV histograms of proteins feature significantly broader distributions than that of NPs. This difference is likely attributed to the different properties between NPs and proteins. Different from proteins, both metallic and dielectric NPs are rigid, with spherical shape and uniform distribution of surface charges. By fitting Gaussians to the events at the main polarity side, the acquired mean ΔV values of negatively charged ferritin, insulin, 26 nm PSNP, 10 nm and 5 nm GNPs, and positively charged cyt c and 50 nm PSNP are −1.69±0.89 mV, −0.11±0.06 mV, −0.24±0.08 mV, −0.13±0.02 mV, −0.06±0.02 mV, +1.01±0.48 mV, and +0.27±0.06 mV, respectively. The mean ΔV of NPs is often comparable or even smaller than the smaller proteins. Because the ΔV magnitude depends on the distance, the higher mobility of proteins and strong interaction between proteins and CNE may facilitate them to be closer to the CNE surface and transiently resides on the surface.

Between negative and positive entities, the ΔV histograms of positive entities often show a longer tail in the distribution, which is likely from the stronger interaction events due to the electrostatic attractive force. However, we did not observe systematically bigger mean ΔV for positive entities. The quartz surface near CNE is quickly saturated with strongly adsorbed entities, which preventing others to be adsorbed or very close to the surface.

### Example 3—Detection of the Net Charge of Proteins Modulated by PH

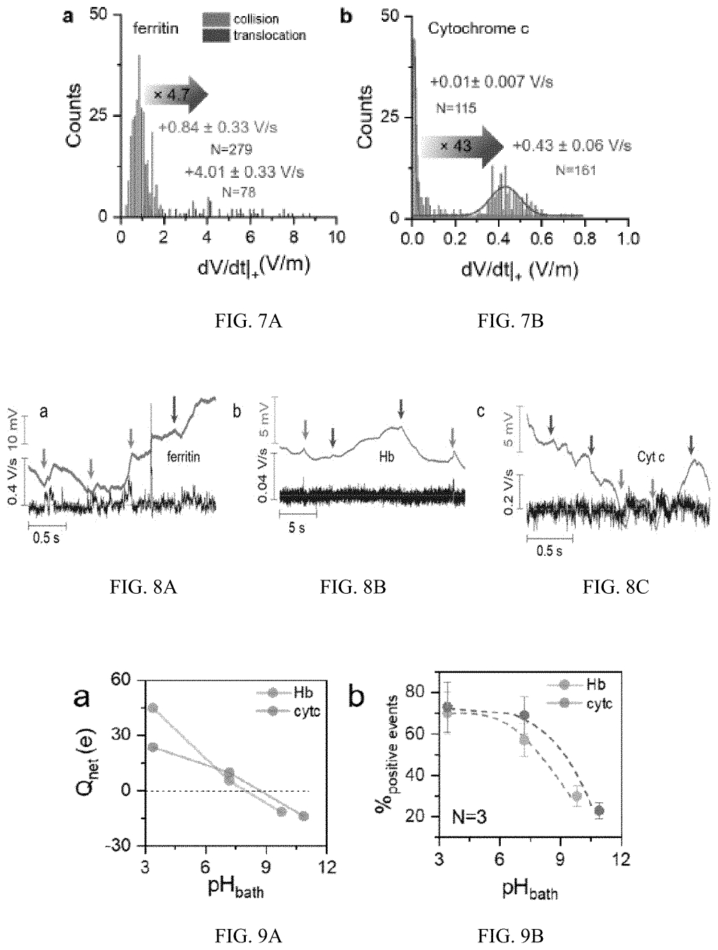

The surface charge of proteins changes with the pH of the environment. To further demonstrate the charge sensing capability of the potentiometric nanoimpact based single protein detection, the solution pH was tuned and the corresponding potential changes of the detected events were monitored. Smaller Cyt c and Hb proteins of similar size were focused on the experiments. Both proteins are stable over the pH range ˜3-11. FIG. 9A shows the estimated net charge change of both proteins with pH. Their net charges become negative at high pH. The potential baseline is not affected by the pH change of the bath solution.

To minimize the effect of nanopipette surface adsorption on potential change, we only analyzed the first 10 min of V-t time traces. The potential shape and amplitude did not change noticeably. The nanopipettes P5 and P6 were used to acquire the nanoimpact events at different pH for Hb and cyt c, respectively. Their nanopore size is about 2-3 times bigger than the protein, thus the translocation events are rare.

Other than a small fraction of undecided events, there are always positive and negative types of nanoimpact events for both Hb and cyt c. As shown in FIG. 9B, for both Hb and cyt c, the positive type events dominate at acidic and neutral pH but their fractions significantly drop at high pH around 10, revealing the incased number of proteins with a positive charge at high pH. This is consistent with the estimated pH-dependent surface charge change of both proteins as shown in FIG. 9A. The results confirm that the potentiometric nanoimpact method can detect the surface charges of protein in the electrolyte solution. Between cyt c and Hb, more positive type nanoimpact events were also observed at pH 3.4 and 7.2 for cyt c. Specially, at pH 7.3, the cyt c has ˜18% more positive events than Hb through their net charge is similar. The difference can be attributed to the relatively larger (˜28%) dipole moment of cyt c than Hb.

FIG. 9C-9D show the nominalized bar histograms of ΔV of dominant nanoimpact event type at three different pH conditions for Hb and cyt c. Based on the Gaussian fits of ΔV histograms, the mean ΔV at each pH condition is calculated and shown as a bar chart adjacent to the histograms. The solid dots in the bar chart in FIG. 9C-9D denote the mean ΔV. Same as FIG. 9B, the major type events give negative mean ΔV at high pH for both proteins, reflecting their negative net surface charge at high pH. At pH 3.4, the ΔV magnitude of Hb is bigger, which can be attributed to the bigger net charge of Hb at low pH. The much broader ΔV distribution of Hb was also observed at pH 3.4 and 7.2 than cyt c. One explanation is that the Hb is more flexible than the cyt c at both pH, as supported by the MD simulation results at pH 7.2. In contrast, the ΔV distribution becomes broader at pH 10.9 for cyt c but becomes narrower at pH 9.8 for Hb. This may suggest that at high pH, cyt c becomes more flexible but Hb becomes more rigid.

### Example 4—Other Type of Nanoelectrodes and Nanoelectrode Surface Modification Procedures for Achieving Higher Specificity in Diseases Biomarkers Detection

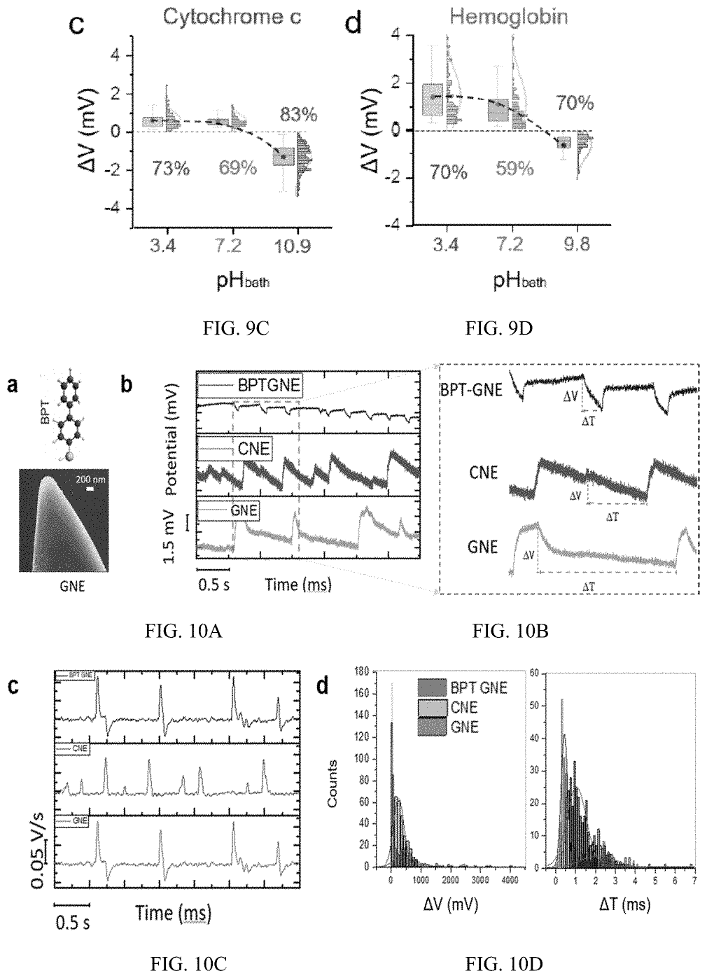

The nanoelectrode can be fabricated by different methods and different conductive materials. FIGS. 10A-10D show the results of potentiometric sensing of GNPs from a CNE made by a single-barrel nanopipette (without the nanopore barrel) and a gold nanoelectrode (GNE) electrochemically etched from a gold wire (0.2 mm diameter) and partially insulated by polymer. Gold can also be electrochemically deposited into the nanopore barrel of the nanopipette to form the GNE. It also shows the nanoelectrode can be used alone without the neighboring nanopore to detect the nanoimpact events of individual entities.

For practical applications, the nanoelectrode surface can be chemically modified to use molecular recognition to avoid fouling and enhance its selectivity. For the surface modification of CNE, reduction of diazonium salt can be used to introduce recognition molecules to the carbon surface. For the surface modification on the glass/quartz, salinization chemistry will be used.

To functionalize the GNE, thiol molecules can be used. In one example, the GNE is immersed in biphenyl-4-thiol (BPT) molecule solution (1 mM in ethanol) for several hours (e.g., 4-8 hrs) to form a self-assembled molecule monolayer. FIGS. 10A-10D show the comparison of transient potential signals generated by collision events of 40 nm GNPs on bare CNE, bare GNE, and BPT molecule modified GNE. The recognition molecules can also mix poly(ethylene glycol) (PEG) or zwitterion molecules to create the stealth effect to avoid non-specific adsorptions on the GNE surface.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A method for a label free detection of a biomolecule in a sample, comprising contacting the sample with a system comprising a nanoelectrode, and measuring a transient electrical potential change induced by a collision event of the biomolecule on the floating nanoelectrode.

2. The method of claim 1, the biomolecule being a charged protein having a molecular weight from about 5 kDa to 500 kDa.

3. The method of claim 1, the sample being a diluted biological sample from a subject.

4. The method of claim 1, the nanoelectrode being made of carbon, gold, copper, titanium, palladium, silver, or platinum.

5. The method of claim 1, the nanoelectrode being made from a single-barrel or double-barrel nanopipettes, or electrochemically etched tips from metal wires.

6. The method of claim 1, the nanoelectrode having an effective area of 0.02-0.30 μm².

7. The method of claim 1, the system comprising a double-barrel nanopipette sensor, the double-barrel nanopipette sensor comprising a first compartment, a second compartment, and a double-barrel nanopipette connecting the first and second compartments, the double-barrel nanopipette having a nanoelectrode barrel and a nanopore barrel, and the nanopore barrel being disposed with an electrode, and the nanoelectrode barrel being filled with the solid nanoelectrode.

8. The method of claim 7, the nanopore barrel having a nanopore at the tip, the nanopore having a diameter from about 10 to 90 nm.

9. The method of claim 7, the method further comprising measuring an ionic current through the nanopore barrel.

10. A method for detecting, quantifying and/or characterizing a protein at a single-molecule level in a sample solution, comprising:
providing a double-barrel nanopipette sensor comprising a double-barrel nanopipette, the double-barrel nanopipette comprising a nanoelectrode barrel and a nanopore barrel, the nanopore barrel being disposed with an electrode;
contacting the double-barrel nanopipette sensor with the sample solution;
optionally, applying a potential through the nanopore barrel, and
measuring one or more signals generated upon the interaction between the protein and the nanopipette, the one or more signals generated being an ionic current through the nanopore barrel and/or a potential change of the nanoelectrode.

11. The method of claim 10, the sample being a biological sample from a subject.

12. The method of claim 10, the nanopore barrel having a nanopore at the tip, the nanopore having a diameter from about 10 to 90 nm.

13. The method of claim 10, the nanoelectrode barrel comprising a nanoelectrode having an effective area of 0.02-0.3 μm².

14. The method of claim 10, the nanoelectrode being made of copper, titanium, palladium, silver, platinum, gold or carbon.

15. The method of claim 10, the nanoelectrode being surface-modified with chemicals to improve protein detection specificity and sensitivity, and avoid fouling.

16. The method of claim 10, the protein being a charged protein having a molecular weight from about 5 kDa to 500 kDa.

17. A microfluidic device for detecting, quantifying and/or characterizing a biomolecule at a single-molecule level, comprising a microfluidic channel in a substrate and a nanoelectrode disposed in the microfluidic channel.

18. The microfluidic device of claim 17, the nanoelectrode being a CNE made from a single-barrel nanopipette or double-barrel nanopipette, or electrochemically etched gold nanoelectrode.

19. The microfluidic device of claim 17, the substrate being selected from PDMS poly(methyl methacrylate) (PMMA), polycarbonate, polystyrene, poly(ethylene glycol) diacrylate (PEGDA), cyclic olefin copolymer, and cyclic olefin polymer (COP).

20. A method for detecting, quantifying and/or characterizing a biomolecule at a single-molecule level in a sample solution, comprising passing the sample solution through the microfluidic device of claim 17, and measuring one or more signals generated upon the interaction between the biomolecule and the nanoelectrode.
